(19) United States
(12) Patent Application Publication
(10) Pub. No.: US 2003/0176351 A1
(43) Pub. Date: Sep. 18, 2003

(54) Peptides and peptide analogues designed from a diabetes-associated autoantigen, and methods for their use in the treatment and prevention of diabetes

(76) Inventors: Gerald T. Nepom, Bainbridge Island, WA (US); Susan Masewicz, Seattle, WA (US); Barbara S. Nepom, Bainbridge Island, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/403,980

(22) Filed: Mar. 28, 2003

Related U.S. Application Data
(63) Continuation of application No. 09/379,211, filed on Aug. 23, 1999.

(51) Int. Cl. A61K 38/10; C07K 7/06; C07K 7/08

(52) U.S. Cl. 514/13; 530/326

(57) ABSTRACT

The present invention relates to peptides and peptide analogues designed from a human pancreatic islet beta cell autoantigen GAD65. In particular, it relates to antagonistic peptides and peptide analogues that antagonize autoimmune T cell activation in response to GAD65. The invention also relates to methods of using such peptides and peptide analogues for the treatment and prevention of type I diabetes or pre-diabetes.

### 1. STATEMENT AS TO RIGHTS TO INVENTIONS MADE UNDER FEDERALLY SPONSORED RESEARCH OR DEVELOPMENT

This invention is made, in part, by government support under grant P01 DK 49841 awarded by the National Institutes of Health. The government may have certain rights in this invention.

### 2. INTRODUCTION

The present invention relates to peptides and peptide analogues designed from a human pancreatic islet beta cell autoantigen GAD65. In particular, it relates to antagonistic peptides and peptide analogues that antagonize autoimmune T cell activation in response to GAD65. The invention also relates to methods of using such peptides and peptide analogues for the treatment and prevention of type I diabetes or pre-diabetes.

### 3. BACKGROUND OF THE INVENTION

Type I diabetes, like many autoimmune diseases, exhibits exquisite target organ specificity, with immune mediated destruction of beta cells in the pancreatic islet, coincident with sparing of the neighboring alpha and delta cells. The precise target cell specificity in this disease implies the existence of antigenic self proteins derived from the beta cell which are specifically recognized by autoimmune T lymphocytes. Extensive analysis of serum antibodies in patients with type I diabetes has documented several self proteins which are candidates for this role (Mehta and Palmer, 1996, Prediction, Prevention and Genetic Counseling in IDDM, John Wiley & Sons, Chichester, Pa.; Gianani and Eisenbarth, 1996, Molecular, Cellular, and Clinical Immunology, Oxford University Press, New York; Nepom, 1995, Curr. Opin. Immunol. 7:825). GAD65, the 65 Kd isoform of glutamic acid decarboxylase, is such a molecule. It is present in pancreatic beta cells at high levels, and antibodies to GAD65 are present in up to 70% of newly diagnosed diabetics (Lernmark, 1996, J. Int. Med. 240:259). Antibodies to GAD65 are also often present for several years prior to the development of clinical diabetes, providing a useful serum marker for prediction of disease onset (Mehta and Palmer, 1996, Prediction, Prevention and Genetic Counseling in IDDM, John Wiley & Sons, Chichester, Pa.; Gianani and Eisenbarth, 1996, Molecular, Cellular, and Clinical Immunology, Oxford University Press, New York; Nepom, 1995, Curr.Opin.Immunol. 7:825; Lernmark, 1996, J. Int. Med. 240:259).

Studies of T cell reactivity to GAD65 in diabetics have confirmed the immunogenicity of this protein, with reports of both CD4⁺ and CD8⁺ T cell responses (Lohmann et al, 1994, Lancet 343:1607; Atkinson et al, 1994, J. Clin. Invest. 94:2125; Armstrong and Jones, 1994, Lancet 344:406; Worsaae et al, 1995, Autoimmunity 22:183; Panina-Bordignon et al, 1995, J. Exp. Med. 181:1923; Endl et al, 1997, J. Clin. Invest. 99:2405; Weiss et al, 1995, Scand. J. Immunol. 42:673; Schloot et al, 1997, Diabetologia 40:332; Bach et al, 1997, J. Autoimmun. 10:375). A diverse array of specificities within the GAD65 protein have been identified, using peptide fragments and synthetic peptides to stimulate human T cell proliferative responses. In general, there is variability within diabetic patients, with recognition of multiple peptides likely related in part to the utilization of multiple major histocompatibility complex (MHC) restriction elements.

For some autoantigens, such as myelin basic protein (MBP) in patients with multiple sclerosis (MS), the array of antigenic peptides appears to be closely restricted by the MHC class II elements which are genetically associated with disease (Gauthier et al, 1998, Proc. Natl. Acad. Sci. USA 95:11828; Smith et al, 1998, J. Exp. Med. 188:1511). For example, the MBP determinant 84-102, which binds to the disease-associated DRB1*1501 and DRB5*0101 alleles, has been described as an immunodominant antigenic specificity for T cells recovered from MS patients (Wucherpfennig et al, 1994, J. Exp. Med. 179:279; Salvetti et al, 1993, Eur. J. Immunol. 23:1232; Valli et al, 1993, J. Clin. Invest. 91:616). In these studies, synthetic peptides containing the stimulatory peptide sequence are used to recall proliferative responses among T cell lines and clones derived from autoimmune patients. The sequence of the epitope is inferred from the most stimulatory synthetic peptide, although the natural epitope is not directly identified.

The MHC haplotypes associated with insulin-dependent diabetes mellitus (IDDM) are well known. Among class II HLA alleles, the DR4 specificity corresponding to the DRB1*0401, *0404, and 0405 alleles is the predominant HLA-DR type expressed in patients, present in approximately 70% of Caucasoid diabetics. These DR4-positive alleles are linked to the DQB1*0302 gene, the HLA-DQ marker most highly associated with IDDM (Nepom and Erlich, 1991, Ann. Rev. Immunol. 9:493). In studies of patients with these HLA disease-susceptibility haplotypes, T cell responses to the GAD65 protein have been documented which are restricted by HLA-DR molecules, indicating the capacity for presenting autoantigenic epitopes from GAD65 for T cell recognition (Lohmann et al, 1994, Lancet 343:1607; Atkinson et al, 1994, J. Clin. Invest. 94:2125; Armstrong and Jones, 1994, Lancet 344:406; Worsaae et al, 1995, Autoimmunity 22:183; Panina-Bordignon et al, 1995, J. Exp. Med. 181:1923; Endl et al, 1997, J. Clin. Invest. 99:2405; Weiss et al, 1995, Scand. J. Immunol. 42:673; Schloot et al, 1997, Diabetologia 40:332; Bach et al, 1997, J. Autoimmun. 10:375). Using a series of overlapping synthetic peptides from the entire GAD65 sequence, previous studies documented approximately 10 peptides which were capable of efficient binding to DR4 molecules, and which therefore were candidates for relevant epitopes likely to be restricted by DR4 and presented for T cell recognition (Wicker et al, 1996, J. Clin. Invest. 98:2597). Indeed, three of these peptides were found to be immunogenic when used to immunize mice transgenic for HLA-DR4, corresponding to epitopes from residues 115-127, 274-286, and 554-566 of human GAD65. A separate study, also using DR4 transgenic mice, found that these same three epitopes were also included in immunodominant regions (116-130, 271-285, and 551-565) when the GAD65 protein, rather than the peptides, was used as the immunogen (Patel et al, 1997, Proc. Natl. Acad. Sci. USA 94:8082).

However, prior to the present invention, it was not known if any of these epitopes were naturally processed by antigen presenting cells (APC) and presented to autoimmune T cells during disease development. More importantly, it was not known in the art how a naturally processed T cell epitope could be modified to produce an antagonistic peptide. Thus, there remains the need to identify diabetes-associated autoantigenic epitopes, and to use them as the basis for the rational design of therapeutic agents for the treatment of IDDM.

### 4. SUMMARY OF THE INVENTION

The present invention relates to peptides and peptide analogues designed from GAD65. In particular, it relates to peptides and peptide analogues that antagonize T cell activation in response to GAD65, pharmaceutical compositions of such peptides and peptide analogues, methods for designing peptides and peptide analogues with similar biologic activities, and methods of using the same to treat or prevent IDDM.

The invention is based, in part, on Applicants' discovery of a major immunodominant epitope of human pancreatic islet antigen GAD65, which is naturally processed by human APC. Such epitope is recognized by human T cell clones which display variable cytokine responses. Synthetic peptides encompassing this epitope stimulated human GAD65-specific T-cells from a DR4-positive individual at high risk of developing IDDM. However, proliferative and cytokine responses by T cell clones recognizing this epitope were antagonized by altered peptide ligands containing a single amino acid modification.

Generally, a compound of the invention is a peptide or peptide analogue of at least 9 amino acids in length. In embodiments wherein the compound is a peptide, it comprises an amino acid sequence that corresponds in primary sequence to GAD65 residues #555-567 which contains at least one amino acid substitution. Such substitution produces a peptide that retains its binding affinity for HLA-DR molecules but does not activate antigen-specific autoimmune T cells. In a preferred embodiment of the invention, the amino acid residue Ile at position 561 is substituted with Met or Leu. In other embodiments, one or more of the other amino acid residues within the peptide are substituted with other conservative amino acid residues, i.e., the amino acid residues are replaced with other amino acid residues having similar physical and/or chemical properties. In embodiments wherein the compound is a peptide analogue, the analogue is obtained by replacing at least one amide linkage in the peptide with a substituted amide or isostere of amide.

In an illustrative embodiment, a compound of the invention comprises the following formula:

Z₁-X₁-X₂-X₃-X₄-X₅-X₆-X₇-X₈-X₉-X₁₀-X₁₁-X₁₂-X₁₃-Z₂ (I)

wherein:

X₁ is absent or any residue;

X₂ is absent or any residue;

X₃ is an aromatic or aliphatic residue;

X₄ is a basic residue;

X₅ is an apolar residue;

X₆ is an aliphatic residue;

X₇ is Met or Leu;

X₈ is a polar residue;

X₉ is Asn;

X₁₀ is an apolar residue;

X₁₁ is an aliphatic or polar residue;

X₁₂ is absent or any residue;

X₁₃ is absent or any residue;

Z₁ is H₂N—, RHN— or, RRN—;

Z₂ is —C(O)OH, —C(O)R, —C(O)OR, —C(O)NHR, —C(O)NRR where each R is independently (C₁-C₆) alkyl, (C₁-C₆) alkenyl, (C₁-C₆) alkynyl, substituted (C₁-C₆) alkyl, substituted (C₁-C₆) alkenyl or substituted (C₁-C₆) alkynyl; and

“—” is a covalent linkage.

It is an object of the invention to treat a human IDDM patient by administering a therapeutically effective amount of a compound of the invention.

It is also an object of the invention to prevent the development of IDDM in an individual by administering a therapeutically effective amount of a compound of the invention. Generally, such an individual contains detectable anti-GAD65 antibodies in the serum and expresses an HLA disease-susceptibility haplotype.

It is another object of the invention to treat a pre-IDDM patient by administering a therapeutically effective amount of a compound of the invention. Generally, such patient contains detectable anti-GAD65 antibodies in the serum, expresses an HLA disease-susceptibility haplotype and exhibits islet cell destruction or compromised insulin function as measured by an intravenous glucose tolerance test.

It is yet another object of the invention to prevent the recurrence of autoimmune disease in a patient following islet transplantation by administering a therapeutically effective amount of a compound of the invention.

### 5. BRIEF DESCRIPTION OF THE DRAWINGS

FIGS. 1A and 1B. T cell response profiles for human CD4⁺ T cell clones BRI.4-10 and BRI.4-11. Proliferation was measured by thymidine uptake (1A) and gamma-IFN release was determined by specific ELISA (1B). Dashed lines designate BRI.4-10, and solid lines designate BRI.4-11.

FIGS. 2A-2D. T cell response to GAD65 residues #555-567 in the presence of altered peptide ligands. Proliferative responses (2A and 2C) and gamma-interferon release (2B and 2D) are shown for T cell clones BRI.4-10 (2A and 2B) and BRI.4-11 (2C and 2D). Square symbols designate 563Q; right-side up triangles designate 559Z; upside down triangles designate 561M; diamonds designate 561L; and circles designate Tet 830-843. Peptide antagonist sequences are given in Table 3.

### 6. DETAILED DESCRIPTION OF THE INVENTION

A large number of studies have suggested the possibility for rational design of peptide antagonists by altering amino acid residues at T cell receptor (TCR) contact sites within an immunogenic epitope, in order to subtly alter the overall avidity of the TCR-MHC-peptide interaction (Evavold et al, 1993, Immunol. Today 14:602; De Magistris et al, 1992, Cell 68:625). Mechanistically, this altered interaction appears to interfere with the duration of TCR signaling events and therefore interfere with the efficiency of substrate phosphorylation and subsequent intracellular signaling. In this invention, peptide antagonists for the GAD65 555-567 epitope were designed by single amino acid substitutions in a predicted TCR contact site. The altered peptide ligands continued to bind to DR4 molecules, but failed to activate epitope-specific autoimmune T cells. Treatment of DR4-expressing APC with both the GAD65 555-567 epitope and an antagonist peptide resulted in complete blockade of T cell activation.

The present invention relates to peptides and peptide analogues designed from GAD65 residues #555-567 which antagonize T cell activation in response to GAD65. Although the specific procedures and methods described herein are exemplified using several specific peptides, they are merely illustrative for the practice of the invention. Analogous procedures and techniques, as well as functionally equivalent peptides and peptide analogues, as will be apparent to those of skill in the art based on the detailed disclosure provided herein are also encompassed by the invention.

As used herein, the following terms shall have the following meanings:

“Alkyl:” refers to a saturated branched, straight chain or cyclic hydrocarbon radical. Typical alkyl groups include, but are not limited to, methyl, ethyl, propyl, isopropyl, butyl, isobutyl, t-butyl, pentyl, isopentyl, hexyl, and the like. In preferred embodiments, the alkyl groups are (C₁-C₆) alkyl, with (C₁-C₃) being particularly preferred.

“Substituted Alkyl:” refers to an alkyl radical wherein one or more hydrogen atoms are each independently replaced with other substituents.

“Alkenyl:” refers to an unsaturated branched, straight chain or cyclic hydrocarbon radical having at least one carbon-carbon double bond. The radical may be in either the cis or trans conformation about the double bond(s). Typical alkenyl groups include, but are not limited to, ethenyl, propenyl, isopropenyl, butenyl, isobutenyl, tert-butenyl, pentenyl, hexenyl and the like. In preferred embodiments, the alkenyl group is (C₁-C₆) alkenyl, with (C₁-C₃) being particularly preferred.

“Substituted Alkenyl:” refers to an alkenyl radical wherein one or more hydrogen atoms are each independently replaced with other substituents.

“Alkynyl:” refers to an unsaturated branched, straight chain or cyclic hydrocarbon radical having at least one carbon-carbon triple bond. Typical alkynyl groups include, but are not limited to, ethynyl, propynyl, butynyl, isobutynyl, pentynyl, hexynyl and the like. In preferred embodiments, the alkynyl group is (C₁-C₆) alkynyl, with (C₁-C₃) being particularly preferred.

“Substituted Alkynyl:” refers to an alkynyl radical wherein one or more hydrogen atoms are each independently replaced with other substituents.

“Alkoxy:” refers to an —OR radical, where R is alkyl, alkenyl or alkynyl, as defined above.

“Aryl:” refers to an unsaturated cyclic hydrocarbon radical having a conjugated π electron system. Typical aryl groups include, but are not limited to, penta-2,4-diene, phenyl, naphthyl, anthracyl, azulenyl, indacenyl, and the like. In preferred embodiments, the aryl group is (C₅-C₂₀) aryl, with (C₅-C₁₀) being particularly preferred.

“Substituted Aryl:” refers to an aryl radical wherein one or more hydrogen atoms are each independently replaced with other substituents.

“Heteroaryl:” refers to an aryl group wherein one or more of the ring carbon atoms is replaced with another atom such as N, O or S. Typical heteroaryl groups include, but are not limited to, furanyl, thienyl, indolyl, pyrrolyl, pyranyl, pyridyl, pyrimidyl, pyrazyl, pyridazyl, purine, pyrimidine and the like.

“Substituted Heteroaryl:” refers to a heteroaryl radical wherein one or more hydrogen atoms are each independently replaced with other substituents.

6.1. Peptides and Peptide Analogues Designed from Autoantigen GAD65 T Cell Epitope

Generally, a compound of the present invention is a peptide or peptide analogue. In embodiments wherein the compound is a peptide, the peptide corresponds in primary sequence to GAD65 residues #555-567 which contains at least one amino acid substitution. In other embodiments, one or more amino acid residues within the peptide are conservatively substituted with other amino acid residues. In embodiments wherein the compound is a peptide analogue, the analogue is obtained by replacing at least one amide linkage in the peptide with a substituted amide or isostere of amide.

A compound of the invention is illustrated by the following formula:

Z₁-X₁-X₂-X₃-X₄-X₅-X₆-X₇-X₈-X₉-X₁₀-X₁₁-X₁₂-X₁₃-Z₂ (I)

wherein:

X₁ is absent or any residue;

X₂ is absent or any residue;

X₃ is an aromatic or aliphatic residue;

X₄ is a basic residue;

X₅ is an apolar residue;

X₆ is an aliphatic residue;

X₇ is Met or Leu;

X₈ is a polar residue;

X₉ is Asn;

X₁₀ is an apolar residue;

X₁₁ is an aliphatic or polar residue;

X₁₂ is absent or any residue;

X₁₃ is absent or any residue;

Z₁ is H₂N—, RHN— or, RRN—;

Z₂ is —C(O)OH, —C(O)R, —C(O)OR, —C(O)NHR, —C(O)NRR where each R is independently (C₁-C₆) alkyl, (C₁-C₆) alkenyl, (C₁-C₆) alkynyl, substituted (C₁-C₆) alkyl, substituted (C₁-C₆) alkenyl or substituted (C₁-C₆) alkynyl; and

“—” is a covalent linkage.

The designation Xₙ in each case represents an amino acid at specified position in the compound. The amino acid residues may be the genetically encoded L-amino acids, naturally-occurring non-genetically encoded L-amino acids, synthetic L-amino acids, or D-enantiomers of all of the above. The amino acid notations used herein for the twenty genetically encoded L-amino acids and common non-encoded amino acids are conventional and are as follows:

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-alanine | | bAla |
| 2,3-diaminopropionic acid | | Dpr |
| α-aminoisobutyric acid | | Aib |
| N-methylglycine (sarcosine) | | MeGly |
| Ornithine | | Orn |
| Citrulline | | Cit |
| t-butylalanine | | t-BuA |
| t-butylglycine | | t-BuG |
| N-methylisoleucine | | MeIle |
| phenylglycine | | Phg |
| cyclohexylalanine | | Cha |
| norleucine | | Nle |
| naphthylalanine | | Nal |
| Pyridylananine | | |
| 3-benzothienyl alanine | | |
| 4-chlorophenylalanine | | Phe(4-Cl) |
| 2-fluorophenylalanine | | Phe(2-F) |
| 3-fluorophenylalanine | | Phe(3-F) |
| 4-fluorophenylalanine | | Phe(4-F) |
| Penicillamine | | Pen |
| 1,2,3,4-tetrahydro-isoquinoline-3- | | Tic |
| carboxylic acid | | |
| β-2-thienylalanine | | Thi |
| Methionine sulfoxide | | MSO |
| Homoarginine | | hArg |
| N-acetyl lysine | | AcLys |
| 2,4-diamino butyric acid | | Dbu |
| p-aminophenylalanine | | Phe(pNH₂) |
| N-methylvaline | | MeVal |
| Homocysteine | | hCys |
| Homoserine | | hSer |
| ε-amino hexanoic acid | | Aha |
| δ-amino valeric acid | | Ava |
| 2,3-diaminobutyric acid | | Dab |

The compounds that are encompassed within the scope of the invention are partially defined in terms of amino acid residues of designated classes. The amino acids may be generally categorized into two main classes: hydrophilic amino acids and hydrophobic amino acids, depending primarily on the characteristics of the amino acid side chain. These main classes may be further divided into subcategories that more distinctly define the characteristics of the amino acid side chains. For example, hydrophilic amino acids include amino acids having acidic, basic or polar side chains; and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid exhibiting a hydrophobicity of greater than zero according to the normalized consensus hydrophobicity scale of Eisenberg et al. (1984, J. Mol. Biol. 179: 125-142). Examples of genetically encoded hydrophobic amino acids include Pro, Phe, Trp, Met, Ala, Gly, Tyr, Ile, Leu and Val. Examples of non-genetically encoded hydrophobic amino acids include t-BuA.

“Aromatic Amino Acid” refers to a hydrophobic amino acid having a side chain containing at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Examples of genetically encoded aromatic amino acids include Phe, Tyr and Trp. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chloro-phenylalanine, 2-fluorophenylalanine, 3-fluorophenylalanine and 4-fluorophenylalanine.

“Apolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Examples of genetically encoded apolar amino acids include Gly, Leu, Val, Ile, Ala and Met. Examples of non-encoded apolar amino acids include Cha.

“Aliphatic Amino Acid” refers to a hydrophobic amino acid having an aliphatic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid exhibiting a hydrophilicity of less than zero according to the normalized consensus hydrophobicity scale of Eisenberg et al. (1984, J. Mol. Biol. 179: 125-142). Examples of genetically encoded hydrophilic amino acids include Thr, His, Glu, Asn, Gln, Asp, Arg, Ser and Lys. Examples of non-encoded hydrophilic amino acids include Cet and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has one bond in which the pair of electrons shared in common by two atoms is held more closely by one of the atoms. Examples of genetically encoded polar amino acids include Ser, Thr, Asn and Gln. Examples of non-genetically encoded polar amino acids include citrulline, N-acetyl lysine and methionine sulfoxide.

The amino acid residue Cys is unusual in that it can form disulfide bridges with other Cys residues or other sulfanyl-containing amino acids. The ability of Cys residues (and other amino acids with —SH containing side chains) to exist in a peptide in either the reduced free —SH or oxidized disulfide-bridged form affects whether Cys residues contribute net hydrophilic or hydrophobic character to a peptide. While Cys exhibits hydrophobicity of 0.29 according to the normalized consensus scale of Eisenberg et al. (supra), it is understood that Cys is classified as a polar hydrophilic amino acid for the purpose of the present invention. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

As will be appreciated by those having skill in the art, the above classifications are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention may be composed include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,4-diaminobutyric acid (Dbu); p-aminophenylalanine (Phe(pNH₂)); N-methyl valine (MeVal); homocysteine (hCys) and homoserine (hSer). These amino acids also fall conveniently into the categories defined above.

The classifications of the above-described genetically encoded and non-encoded amino acids are summarized in Table 1, below. It is to be understood that Table 1 is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 1

| | Genetically | |
|---|---|---|
| Classification | Encoded | Genetically Non-Encoded |
| Hydrophobic | | |
| Aromatic | F, Y, W | Phg, Nal, Thi, Tic, Phe(4-Cl), Phe(2-F), |
| | | Phe(3-F), Phe(4-F), Pyridyl Ala, |
| | | Benzothienyl Ala |
| Apolar | L, V, I, A, M, | T-BuA, T-BuG, MeIRe, Nle, MeVal, |
| | G, P | Cha, MeGly, Aib |
| Aliphatic | A, V, L, I | t-BuA, t-BuG, MeIle, Nle, MeVal, Cha, |
| | | bAla, MeGly, Aib, Dpr, Aha |
| Hydrophilic | | |
| Acidic | D, E | |
| Basic | H, K, R | Dpr, Orn, hArg, Phe(p-NH₂), Dbu, Dab |
| Polar | C, Q, N, S, T | Cit, AcLys, MSO, hSer, bAla |
| Helix-Breaking | P, G | D-Pro and other D-amino acids (in L- |
| | | peptides) |

In the compounds of formulae (I), the symbol “—” between amino acid residues generally designates a backbone interlinkage. Thus, the symbol “—” usually designates an amide linkage (—C(O)—NH). It is to be understood, however, that in all of the peptides described in the specific embodiments herein, one or more amide linkages may optionally be replaced with a linkage other than amide, preferably a substituted amide or an isostere of an amide linkage. Thus, while the various Xₙ have generally been described in terms of amino acids, one having skill in the art will recognize that in embodiments having non-amide linkages, the term “amino acid” refers to other bifunctional moieties having side-chain groups similar to the side chains of the amino acids. For example, in embodiments having non-amide linkages, the phrase “acidic amino acid” refers to a bifunctional molecule capable of forming the desired backbone interlinkages and which has a side chain group similar to the side chain of an acidic amino acid. Substituted amides generally include groups of the formula —C(O)—NR, where R is (C₁-C₆) alkyl, (C₁-C₆) alkenyl, (C₁-C₆) alkynyl, substituted (C₁-C₆) alkyl, substituted (C₁-C₆) alkenyl or substituted (C₁-C₆) alkynyl. Isosteres of amide generally include, but are not limited to, —CH₂NH—, —CH₂S—, —CH₂CH₂, —CH═CH— (cis and trans), —C(O)CH₂—, —CH(OH)CH₂— and —CH₂SO—.

Compounds having such linkages and methods for preparing such compounds are well-known in the art (see, e.g., Spatola, 1983, Vega Data 1(3) for a general review); Spatola, 1983, “Peptide Backbone Modifications” In: Chemistry and Biochemistry of Amino Acids Peptides and Proteins (Weinstein, ed.), Marcel Dekker, New York, p. 267 (general review); Morley, 1980, Trends Pharm. Sci. 1:463-468; Hudson et al., 1979, Int. J. Prot. Res. 14:177-185 (—CH₂NH—, —CH₂CH₂—); Spatola et al., 1986, Life Sci. 38:1243-1249 (—CH₂—S); Hann, 1982, J. Chem. Soc. Perkin Trans. I. 1:307-314 (—CH═CH—, cis and trans); Almquist et al., 1980, J. Med. Chem. 23:1392-1398 (—COCH₂—); Jennings-White et al., Tetrahedron. Lett. 23:2533 (—COCH₂—); European Patent Application EP 045 665 (1982) CA:97:39405 (—CH(OH)CH₂—); Holladay et al., 1983, Tetrahedron Lett. 24:4401-4404 (—C(OH)CH₂—); and Hruby, 1982, Life Sci. 31:189-199 (—CH₂—S—).

Additionally, the compounds of the invention may have end modifications, denoted as Z₁ and Z₂ in formula (I). Such modifications can contain non-interfering amino acid residues. In one embodiment, the amino acid residue Val may be added to the amino terminus. In another embodiment, the amino acid sequence His-Gln-Asp may be added to the carboxyl terminus.

In a preferred embodiment of the invention, the compounds of formula (I) are defined as follows:

X₁-X₂-X₃-X₄-X₅-X₆-X₇-X₈-X₉-X₁₀-X₁₁-X₁₂-X₁₃

wherein:

X₁ is absent or a polar amino acid;

X₂ is absent or an aromatic amino acid;

X₃ is an aromatic or aliphatic amino acid;

X₄ is Arg or Lys;

X₅ is Met, Ile or Val;

X₆ is an aliphatic amino acid;

X₇ is Met or Leu;

X₈ is Ser or Thr;

X₉ is Asn;

X₁₀ is an apolar amino acid;

X₁₁ is an aliphatic amino acid;

X₁₂ is absent or an aliphatic amino acid;

X₁₃ is absent or a polar amino acid;

“—” is an amide, substituted amide or an isostere of amide thereof.

In a particularly preferred embodiment, the compounds of the invention are those of formula (I) wherein:

X₁ is absent or Asn;

X₂ is absent or Phe;

X₃ is Phe, Tyr, Trp or Ile;

X₄ is Arg or Lys;

X₅ is Met, Ile or Val;

X₆ is Val, Ile, Ala or Leu;

X₇ is Met or Leu;

X₈ is Ser or Thr;

X₉ is Asn;

X₁₀ is Pro, Gly, Ala or Ser;

X₁₁ is Ala or Ser;

X₁₂ is absent or Ala;

X₁₃ is absent or Thr;

Z₁ is H₂N;

Z₂ is —C(O)OH; and

“—” is an amide linkage.

In one preferred embodiment, “—” between each Xₙ is —C(O)NH— or —C(O)NR—, where R is (C₁-C₆) alkyl, (C₂-C₆) alkenyl or (C₂-C₆) alkynyl, preferably (C₁-C₆) alkyl.

In another preferred embodiment, X₇ is Met.

In still another preferred embodiment, X₇ is Leu.

In still another preferred embodiment, X₁, X₂, X₁₂ and X₁₃ are absent.

Particularly preferred peptides of the invention include the following:

| | |
|---|---|
| NFFRMVMSNPAAT; | (SEQ ID NO:1) |
| NFFRMVLSNPAAT; | (SEQ ID NO:2) |
| FFRMVMSNPAA; | (SEQ ID NO:3) |
| FFRMVLSNPAA; | (SEQ ID NO:4) |
| FFRMVMTNPAA; | (SEQ ID NO:5) |
| FFRMVLTNPAA; | (SEQ ID NO:6) |
| FYRMVMSNPAA; | (SEQ ID NO:7) |
| FYRMVLSNPAA; | (SEQ ID NO:8) |
| FYRMVMTNPAA; | (SEQ ID NO:9) |
| FYRMVLTNPAA; | (SEQ ID NO:10) |
| FWRMVMSNPAA; | (SEQ ID NO:11) |
| FWRMVLSNPAA; | (SEQ ID NO:12) |
| FWRMVMTNPAA; | (SEQ ID NO:13) |
| FWRMVLTNPAA; | (SEQ ID NO:14) |
| FRMVMSNPAA; | (SEQ ID NO:15) |
| FRMVLSNPAA; | (SEQ ID NO:16) |
| FRMVMTNPAA; | (SEQ ID NO:17) |
| FRMVLTNPAA; | (SEQ ID NO:18) |
| FRMVMSNPA; | (SEQ ID NO:19) |
| FRMVLSNPA; | (SEQ ID NO:20) |
| FRMVMTNPA; | (SEQ ID NO:21) |
| FRMVLTNPA. | (SEQ ID NO:22) |

In all of the aforementioned embodiments of the invention, it is to be understood that the phrase “amino acid” also refers to bifunctional moieties having amino acid-like side chains, as previously described.

Generally, active peptides or peptide analogues of the invention are those that bind HLA-DR4 molecules and exhibit at least about 15% inhibition of T cell response to GAD65 as measured in vitro assays such as those described in Section 7, infra. Preferably, active peptides of the invention or analogues thereof will exhibit at least about 20% to 50% or even 80% or more inhibition T cell activation in response to GAD65, as measured by T cell proliferation or cytokine production.

6.2. Preparation of Peptides and Peptide Analogues

6.2.1. Chemical Synthesis

The peptides of the invention or analogues thereof, may be prepared using virtually any art-known technique for the preparation of peptides and peptide analogues. For example, the peptides may be prepared in linear form using conventional solution or solid phase peptide syntheses and cleaved from the resin followed by purification procedures (Creighton, 1983, Protein Structures And Molecular Principles, W. H. Freeman and Co., N.Y.). Suitable procedures for synthesizing the peptides described herein are well known in the art. The composition of the synthetic peptides may be confirmed by amino acid analysis or sequencing (e.g., the Edman degradation procedure and mass spectroscopy).

In addition, analogues and derivatives of the peptides can be chemically synthesized. The linkage between each amino acid of the peptides of the invention may be an amide, a substituted amide or an isostere of amide. Nonclassical amino acids or chemical amino acid analogues can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general. Furthermore, the amino acid can be D (dextrorotary) or L (levorotary).

Cyclized peptides may be formed by the addition of Cys residues to the termini of linear peptides. Formation of disulfide linkages, if desired, is generally conducted in the presence of mild oxidizing agents. Chemical oxidizing agents may be used, or the compounds may simply be exposed to atmospheric oxygen to effect these linkages. Various methods are known in the art, including those described, for example, by Tam, J. P. et al., 1979, Synthesis 955-957; Stewart et al., 1984, Solid Phase Peptide Synthesis, 2d Ed., Pierce Chemical Company Rockford, Ill; Ahmed et al., 1975, J. Biol. Chem. 250:8477-8482; and Pennington et al., 1991 Peptides 1990 164-166, Giralt and Andreu, Eds., ESCOM Leiden, The Netherlands. An additional alternative is described by Kamber et al., 1980, Helv Chim Acta 63:899-915. A method conducted on solid supports is described by Albericio, 1985, Int. J. Peptide Protein Res. 26:92-97. Any of these methods may be used to form disulfide linkages in the peptides of the invention.

6.2.2. Recombinant Synthesis

If the peptide is composed entirely of gene-encoded amino acids, or a portion of it is so composed, the peptide or the relevant portion may also be synthesized using conventional recombinant genetic engineering techniques.

For recombinant production, a polynucleotide sequence encoding a linear form of the peptide is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Maniatis et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.). The coding sequence for human GAD65 has been described (Bu et al., 1992, Proc. Natl. Acad. Sci. U.S.A. 89:2115-2119; Bu and Tobin, 1994, Genomics 21:222-228). Methods for introducing codon substitutions to the native sequence in order to encode an antagonistic peptide based on the disclosure herein are well known to those skilled in the art. For example, a preferred coding sequence contains the following nucleotide sequence: AAT TTC TTC CGC ATG GTC ATG TCA AAC CCA GCG GCA ACT (SEQ ID NO: 23) which encodes the peptide of SEQ ID NO: 1.

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of expression product, SV40-, BPV- and EBV-based vectors may be used with an appropriate selectable marker.

In cases where plant expression vectors are used, the expression of sequences encoding the peptides of the invention may be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843) or heat shock promoters, e.g., soybean hspl7.5-E or hspl7.3-B (Gurley et al., 1986, Mol. Cell. Biol. 6:559-565) may be used. These constructs can be introduced into plant cells using Ti plasmids, Ri plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In one insect expression system that may be used to produce the peptides of the invention, Autographa californica nuclear polyhidrosis virus (AcNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (See e.g., Smith et al., 1983, J. Virol. 46:584; Smith, U.S. Pat. No. 4,215,051). Further examples of this expression system may be found in Current Protocols in Molecular Biology, Vol. 2, Ausubel et al., eds., Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts (see e.g., Logan & Shenk, 1984, Proc. Natl. Acad. Sci. USA 81:3655-3659). Alternatively, the vaccinia 7.5 K promoter may be used (see, e.g., Mackett et al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931).

Other expression systems for producing the peptides of the invention will be apparent to those having skill in the art.

6.2.3. Purification Methods

The peptides and peptide analogues of the invention can be purified by art-known techniques such as high performance liquid chromatography, ion exchange chromatography, gel electrophoresis, affinity chromatography and the like. The actual conditions used to purify a particular peptide or analogue will depend, in part, on factors such as net charge, hydrophobicity, hydrophilicity, etc., and will be apparent to those having skill in the art.

For affinity chromatography purification, any antibody which specifically binds the peptides or peptide analogues may be used. For the production of antibodies, various host animals, including but not limited to rabbits, mice, rats, hamsters, etc., may be immunized by injection with a linear peptide. The peptide may be attached to a suitable carrier, such as BSA or KLH, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a peptide may be prepared using any technique which provides for the production of antibody molecules by continuous cell lines in culture. These include but are not limited to the hybridoma technique originally described by Koehler and Milstein, (1975, Nature 256:495-497), the human B-cell hybridoma technique, (Kosbor et al., 1983, Immunology Today 4:72; Cote et al., 1983, Proc. Natl. Acad. Sci. USA 80:2026-2030) and the EBV-hybridoma technique (Cole et al., 1985, Monoclonal Antibodies and Cancer Therapy, Alan R. Liss, Inc., pp. 77-96 (1985)). In addition, techniques developed for the production of “chimeric antibodies” (Morrison et al, 1984, Proc. Natl. Acad. Sci. USA 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce peptide-specific single chain antibodies.

Antibody fragments which contain deletions of specific binding sites may be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. (See, Scopes, 1984, Protein Purification: Principles and Practice, Springer-Verlag New York, Inc., NY, Livingstone, 1974, Methods Enzymology: Immunoaffinity Chromatography of Proteins 34:723-731).

6.3. Uses of Peptide and Peptide Analogues Designed from Autoantigen GAD65 T Cell Epitope

The compounds of the present invention are useful for inhibiting autoimmune T cell activation in response to GAD65 antigen. As a result, the compounds are particularly useful for the treatment or prevention of IDDM. In a preferred embodiment of the invention, a compound of the invention binds to HLA class II molecules but does not activate T cells. Additionally, the compounds of the invention are useful in treating individuals with pre-IDDM. Several specific criteria for determining the condition of pre-IDDM have been described in Diabetes Care, 1999, Volume 22, Supplement 1. In particular, these include hyperglycemia as measured by blood or urine glucose levels, expression of HLA disease haplotype, serum antibodies against GAD65 and islet cell destruction as measured by IVGTT (Srikanta et al., 1984, Diabetes 33:717-720; Perley and Kipnis, 1966, J. Clin. Invest. 46:1954-1962; Brunzell et al., 1976, J. Clin. Endocrinol. Metab. 42:222-229). While individuals who are positive for the aforementioned criteria do not manifest full clinical symptoms of IDDM, such pre-diabetics can be treated with the compounds of the invention to prevent or retard the development of disease.

6.3.1. Formulation and Route of Administration

The compounds of the invention may be administered to a subject per se or in the form of a pharmaceutical composition. Pharmaceutical compositions comprising the compounds of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the active peptides or peptide analogues into preparations which can be used pharmaceutically. Proper formulation is dependent upon the route of administration chosen.

For topical administration the compounds of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration. For injection, the compounds of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the compounds may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the compounds can be readily formulated by combining the active peptides or peptide analogues with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compounds may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may also be formulated in rectal or vaginal compositions such as suppositories or retention enemas, e.g, containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

6.3.2. Effective Dosages

The compounds of the invention will generally be used in an amount effective to achieve the intended purpose. For use to treat or prevent IDDM, the compounds of the invention, or pharmaceutical compositions thereof, are administered or applied in a therapeutically effective amount. By therapeutically effective amount is meant an amount effective to ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture (i.e., the concentration of test compound that inhibits 50% of T cell/peptide-pulsed APC binding interactions or 50% T cell activation). Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs.

6.3.3. Toxicity

Preferably, a therapeutically effective dose of the compounds described herein will provide therapeutic benefit without causing substantial toxicity.

Toxicity of the compounds described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1996, In: The Pharmacological Basis of Therapeutics, 9^{th} ed., Chapter 2, p. 29, Elliot M. Ross).

The invention having been described, the following examples are offered by way of illustration and not limitation.

### 7. EXAMPLE

### Antagonistic Peptides Inhibited T Cell Activation in Response to a Naturally Processed GAD65 Epitope

7.1. Materials and Methods

7.1.1. Patient Selection

Newly diagnosed IDDM patients between the ages of 14 and 25, treated for diabetes at the Virginia Mason Medical Center Section of Endocrinology, were asked to participate in the study. All participating patients were typed for HLA class II DR and DQ alleles, and serum was tested for autoantibodies to hGAD65, insulin, and IA-2, using standard protocols. Patients who were DR4-positive and who had autoantibodies to GAD65 were selected for T cell analysis. A non-diabetic individual initially identified as positive for autoantibodies to ICA, GAD65, and IA2 in an on-going serum screening project, HLA-DR4 [HLA-DRB1*0404,*0405; HLA-DQB1*0302,*0302], was also studied. Using the criteria of a high-risk HLA genotype and two or more anti-islet autoantibodies, this individual was defined as at-risk for IDDM. Intravenous glucose tolerance test (IVGTT) assays were performed at the same time as T cell studies were initiated, and were within normal limits (Srikanta et al., 1984, Diabetes 33:717-720). This patient continues to be followed in a pre-diabetes screening program at Virginia Mason Research Center.

7.1.2. Proliferation and Cytokine Production Assays

10⁵ thawed and irradiated DRB1*0404, DRB1*0405, or control (non-DR4) peripheral blood lymphocytes (PBL) in a volume of 100 μl were added to wells of 96 V-bottom plates containing peptide or medium and allowed to incubate for 2-3 hours at which time 4×10⁴ T cells were added for a total volume of 200 μl-250 μl. At 20-24 hours of coculture, supernatants were harvested for cytokine determination and the wells replenished with fresh medium. At 48 hours, wells were radiolabeled with 1 μCi H³-thymidine and cultured for an additional 18 hr. The plates were harvested on a TomTec Manual Mach III harvester and incorporated thymidine (as cpm) was determined by liquid spectroscopy on a Wallac Microbeta LSC.

For prepulse assays, APC were preincubated for 2-3 hours with suboptimal concentrations of the agonist peptide, washed 3 times, then cultured with the antagonist peptides.

Traditional sandwich ELISA were performed to test the supernatants for human γIFN, using matched antibody sets obtained from Endogen. The plates were read at 405 nm on a Microplate reader (Bio-Tek). The concentration of cytokine was estimated from standard curves using linear regression.

7.1.3. Generation of T Cell Clones

PBL were primed for 10 days with a 10 μg/ml pool of peptides spanning the C-terminus of human GAD65 antigen. At day 10 of culture, T cells were plated at 0.3, 3, 10 cells/well together with 10⁴ irradiated, autologous, GAD-pulsed PBL in 10 μl of IL2 and IL7 supplemented conditioned medium in sterile Terasaki plates. Conditioned medium consisted of RPMI-1640 supplemented with 2 mM L-glutamine, 100 μg/ml penicillin/streptomycin, 1 mM sodium pyruvate, 15% v/v pooled human serum (PHS) obtained from 20-25 healthy, untransfused male donors. After 10-14 days of incubation in a 37° C., 5% CO₂ atmosphere, wells having positive growth were transferred to 96 well flat bottom plates containing 10⁵ irradiated autologous GAD 555-567 peptide-pulsed PBL, 10 μg/ml IL2 (Intergen), 10 ng/ml IL7 (Pharmingen), and 0.4 μg/ml PHA (SIGMA). After another 14 days of culture, all wells were assayed for specificity to GAD 555-567 measuring both H³-thymidine uptake and γIFN production. Wells of interest were further expanded with autologous PBL plus supplemented conditioned medium as described above. The restriction elements were determined by testing an APC panel of BLS-1 cells transfected with HLA class II genes representative of the donor's DR type, i.e., BLS DRB1*0404, *0405 and DRB4*0101. All T cell clones in this study were found to recognize GAD 555-567 in the context of both DRB1*0404 and *0405 gene products and not DRB4 encoded molecules.

7.1.4. Peptide Synthesis

Peptides used for T cell stimulation and MHC binding studies were synthesized with an Applied Biosystems 432 Peptide Synthesizer (Foster City, Calif.). Binding assays were performed as described by Kwok et al. (1995, J. Immunol. 155:2468).

7.2. Results

In order to determine the relevance of candidate epitopes to the human immune response, T cell recognition of GAD65 epitopes was studied in the context of the DR4 restriction element, utilizing T cells and APC from HLA-DR4-positive individuals. Eight patients positive for HLA-DR4, with recent-onset IDDM (less than 15 months post-diagnosis), were tested for T cell responses to a panel of peptides from GAD65 which encompassed a set of candidate epitopes previously identified in studies using DR4 transgenic mice (Wicker et al, 1996, J. Clin. Invest. 98:2597; Patel et al, 1997, Proc. Natl. Acad. Sci. USA 94:8082). One of the most immunodominant of these epitopes corresponded to the region near the carboxy-terminus of GAD65, represented in the antigen panel by peptides from the region encompassing residues #553-585.

The γIFN cytokine response for these patients, measured in supernatants from antigen-stimulated T cells cultured with peptide-pulsed autologous APC, is shown in Table 2.

TABLE 2
T cell reactivity to peptides from
GAD65 residues #553-585 in DR4-positive patients

| Subject | HLA-DRB1 | Months | γIFN | IL4 | GAD65 Ab |
|---|---|---|---|---|---|
| I.D. | Typing | After Diagnosis | pg/ml | pg/ml | index |
| 6118 | 0301, 0404 | 5 | 0 | 0 | 0.24 |
| 6544 | 0401, 0401 | 1 | 6 | 0 | 1.1 |
| 6616 | 0301, 0401 | 14 | 79 | 0 | 0 |
| 6545 | 0301, 0404 | 1.5 | 120 | 0 | 0.94 |
| 6862 | 0301, 0404 | 1.5 | 176 | 0 | 0.37 |
| 6815 | 0301, 0401 | 1.5 | 264 | 0 | 0.07 |
| 6434 | 0401, 0404 | 0.8 | 376 | 0 | 0.2 |
| 7417 | 0401, 0101 | 0.5 | 4 | 0 | 0.55 |
| 6211 | 0404, 0405 | — | 261 | 0 | 0.18 |

Five of eight HLA-DR4 patients tested showed robust γIFN output after stimulation; specificity of this response was verified by lack of stimulation with other GAD65 peptides in the same experiment for each patient. Also shown in Table 2 is the T cell response for patient #6211, a non-diabetic HLA-DR4 individual at risk for IDDM, who also had strong γIFN cytokine responses to peptides from this GAD65 region. No IL-4 was detected in any of the cultures.

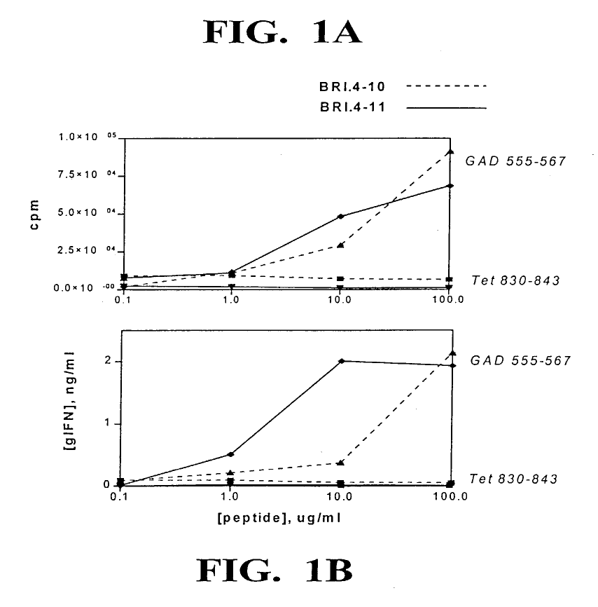

T cells from Patient #6211 were expanded in serial culture by restimulation with GAD65 peptides incubated with autologous APC. Specific T cell responses were present for both peptides 553-572 (53 pg/ml γIFN) and 555-567 (51 pg/ml γIFN), but not peptide 569-585 (5 pg/ml γIFN), localizing the minimal epitope to the 555-567 region. T cell clones were derived by expansion of this culture; proliferation, and cytokine response profiles for CD4⁺ T cell clones BRI.4-10 and BRI.4-11 are shown in FIGS. 1A and 1B.

Human GAD65 gene was transfected into DR4-homozygous B cell lines. The naturally processed GAD65 peptides bound by DR4 were analyzed by nanoflow HPLC interfaced with electrospray ionization mass spectrometry. The sequence of GAD65 residues #554-570 was determined to be a native autoantigen, processed and presented by human APC to human peripheral T cells from an individual at risk for IDDM. In a detailed study of potential DR4-binding peptides derived from GAD65, the 554-566 sequence was the most avid binding peptide identified (Wicker et al, 1996, J. Clin. Invest. 98:2597). Residues within this sequence contain a prototypic DR4-binding motif, in which specific residues are suitable for anchor positions 1,4,6 and 9-corresponding to the four main side chain binding pockets in the DR4 molecule (Hill et al, 1994, J. Immunol. 152:2890; Hammer et al, 1994, Proc. Natl. Acad. Sci. USA 91:4456; Sette et al, 1993, J. Immunol. 151:3163; Dessen et al, 1997, Immunity 7:473). This potential motif occurs within the GAD 554-570 sequence VNFFRMVISNPAATHQD (SEQ ID NO: 24), predicting a class II binding motif in which F-557, V-560, S-562, and A-565 correspond to the P1, P4, P6 and P9 anchors. Table 3A shows the sequences of alanine substituted analogs which were synthesized in order to validate this motif by modifying the likely P1 anchor residue. Binding of peptides to DR4 molecules was diminished by alanine substitution of the F-557 residue.

TABLE 3

| | |
|---|---|
| peptide sequences | peptide binding¹ |

| | | | |
|---|---|---|---|
| | GAD 554-570 | VNFFRMVISNPAATHQD (SEQ ID NO: 24) | 0.6 μM |
| | | 1 4 6 9 | |
| A. | 555-567 | NFFRMVISNPAAT (SEQ ID NO: 25) | 0.7 μM |
| | 557 A | --A---------- (SEQ ID NO:26) | >1 μM |
| | 556 A | -A----------- (SEQ ID NO:27) | 0.6 μM |
| B. | 559 Nle | ----Z-------- (SEQ ID NO:28) | 0.3 μM |
| | 561 M | ------M------ (SEQ ID NO:1) | 0.4 μM |
| | 561 L | ------L------ (SEQ ID NO:2) | 0.4 μM |
| | 563 Q | --------Q---- (SEQ ID NO:29) | 0.5 μM |

¹Concentration of peptide giving 50% inhibition of a standard DR4-peptide fluorescent binding assay

Based on this motif, additional peptide analogs were synthesized in which putative T cell contact residues on the peptide were modified. Substitutions at P3 (methionine to norleucine at GAD65 residue #559), P5 (isoleucine to methionine or leucine at GAD65 residue #561), and P7 (asparagine to glutamine at GAD65 residue #563) were introduced, in which fairly conserved changes to the likely T cell interaction sites were intended to alter the strength of antigenic signal delivered for TCR recognition without changing the class II binding profile. As expected, peptides containing each of these substitutions were found to bind to DR4 class II molecules comparable to the unmodified sequence. These peptides are listed in Table 3B. Each of these modified peptides were also tested for ability to trigger proliferation or γIFN release from T cell clones BRI.4-10 and BRI.4-11; no T cell stimulation was observed, consistent with the predicted loss of agonist activity by changes at TCR contact residues of the peptide epitope.

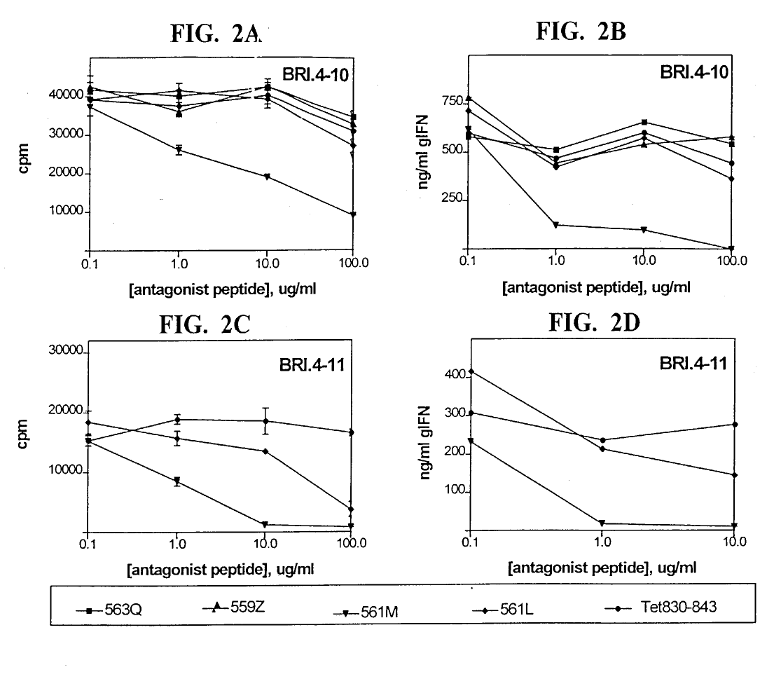

FIGS. 2A-2D illustrate T cell responses to the GAD65 residues #555-567 peptide in the presence of peptides containing substitutions at T cell contact sites. In these assays, APC are pre-pulsed with the agonist peptide, so that reduction in T cell responses indicates antagonism resulting from exposure to the modified peptides. Methionine substitution at P5 resulted in significant antagonism of the antigen-specific T cell response. The T cell proliferative response was reduced by 80% for T cell clone BRI.4-10 when incubated with the 561M antagonist peptide. A control peptide derived from tetanus toxin 830-843, as well as the P3 and P7 substituted peptides, had no effect. The γIFN cytokine response of clone BRI.4-10 was similarly antagonized, with a much greater sensitivity to the 561M APL (FIG. 2B). The 561M APL also antagonized T cell responses of clone BRI.4-11 by more than 90% (FIGS. 2C and 2D). In addition, the other P5 substitution, 561L, partially antagonized the proliferative response of T cell clone BRI.4-11 at the highest concentration tested.

The present invention is not to be limited in scope by the exemplified embodiments which are intended as illustrations of single aspects of the invention and any sequences which are functionally equivalent are within the scope of the invention. Indeed, various modifications of the invention in addition to those shown and described herein will become apparent to those skilled in the art from the foregoing description and accompanying drawings. Such modifications are intended to fall within the scope of the appended claims. All publications cited herein are incorporated by reference in their entirety.

What is claimed is:

1. A compound having the formula:
Z₁-X₁-X₂-X₃-X₄-X₅-X₆-X₇-X₈-X₉-X₁₀-X₁₁-X₁₂-X₁₃-Z₂. (I)
wherein:
X₁ is absent or any residue;
X₂ is absent or any residue;
X₃ is an aromatic or aliphatic residue;
X₄ is a basic residue;
X₅ is an apolar residue;
X₆ is an aliphatic residue;
X₇ is Met or Leu;
X₈ is a polar residue;
X₉ is Asn;
X₁₀ is an apolar residue;
X₁₁ is an aliphatic or polar residue;
X₁₂ is absent or any residue;
X₁₃ is absent or any residue;
Z₁ is H₂N—, RHN— or, RRN—;
Z₂ is —C(O)R, —C(O)OR, —C(O)NHR, —C(O)NRR where each R is independently (C₁-C₆) alkyl, (C₁-C₆) alkenyl, (C₁-C₆) alkynyl, substituted (C₁-C₆) alkyl, substituted (C₁-C₆) alkenyl or substituted (C₁-C₆) alkynyl; and
“—” is a covalent linkage.

2. The compound of claim 1 in which X₁ is absent or a polar amino acid; X₂ is absent or an aromatic amino acid; X₃ is an aromatic or aliphatic amino acid; X₄ is Arg or Lys; X₅ is Met, Ile or Val; X₆ is an aliphatic amino acid; X₇ is Met or Leu; X₈ is Ser or Thr; X₉ is Asn; X₁₀ is an apolar amino acid; X₁₁ is an aliphatic amino acid; X₁₂ is absent or an aliphatic amino acid; X₁₃ is absent or a polar amino acid; and “—” is an amide, substituted amide or an isostere of amide thereof.

3. The compound of claim 2 in which X₁ is absent or Asn; X₂ is Phe or absent; X₃ is Phe, Tyr, Trp or Ile; X₄ is Arg or Lys; X₅ is Met, Val or Ile; X₆ is Val, Ile, Ala or Leu; X₇ is Met or Leu; X₈ is Ser or Thr; X₉ is Asn; X₁₀ is Pro, Gly, Ala or Ser; X₁₁ is Ala or Ser; X₁₂ is absent or Ala; X₁₃ is absent or Thr; Z₁ is H₂N; Z₂ is —C(O)OH; and “—” is an amide linkage.

4. The compound of claim 3 in which the compound is selected from the group consisting of SEQ ID NOS: 1-22.

5. A pharmaceutical composition, comprising the compound of claim 1 and a pharmaceutical excipient carrier or an excipient.

6. A method of inhibiting T cell activation in response to GAD65, comprising contacting a T cell with an effective amount of the compound of claim 1.

7. A method of treating IDDM, comprising administering to a subject a therapeutically effective amount of the compound of claim 1.

8. A method of preventing IDDM, comprising administering to a subject a therapeutically effective amount of the compound of claim 1.

9. A method of treating pre-IDDM, comprising administering to a subject a therapeutically effective amount of the compound of claim 1.

10. A method of preventing recurring autoimmunity after islet transplantation, comprising administering to a subject a therapeutically effective amount of the compound of claim 1.


SEQUENCE LISTING (29 sequences)

SEQ ID NO: 1 - protein, 13 aa, Artificial Sequence
  Description of Artificial Sequence HLA-DR4 binding peptide with mutation at residue 7.
NFFRMVMSNPAAT

SEQ ID NO: 2 - protein, 13 aa, Artificial Sequence
  Description of Artificial Sequence HLA-DR4 binding peptide with mutation at residue 7.
NFFRMVLSNPAAT

SEQ ID NO: 3 - protein, 11 aa, Artificial Sequence
  Description of Artificial Sequence  HLA-DR4 binding peptide with mutation at residue 6.
FFRMVMSNPAA

SEQ ID NO: 4 - protein, 11 aa, Artificial Sequence
  Description of Artificial Sequence  HLA-DR4 binding peptide with mutation at residue 6.
FFRMVLSNPAA

SEQ ID NO: 5 - protein, 11 aa, Artificial Sequence
  Description of Artificial Sequence HLA-DR4 binding peptide with mutation at residues 6 and 7.
FFRMVMTNPAA

SEQ ID NO: 6 - protein, 11 aa, Artificial Sequence
  Description of Artificial Sequence HLA-DR4 binding peptide with mutation at residues 6 and 7.
FFRMVLTNPAA

SEQ ID NO: 7 - protein, 11 aa, Artificial Sequence
  Description of Artificial Sequence HLA-DR4 binding peptide with mutation at residues 2 and 6.
FYRMVMSNPAA

SEQ ID NO: 8 - protein, 11 aa, Artificial Sequence
  Description of Artificial Sequence HLA-DR4 binding peptide with mutation at residues 2 and 6.
FYRMVLSNPAA

SEQ ID NO: 9 - protein, 11 aa, Artificial Sequence
  Description of Artificial Sequence HLA-DR4 binding peptide with mutation at residues 2, 6 and 7.
FYRMVMTNPAA

SEQ ID NO: 10 - protein, 11 aa, Artificial Sequence
  Description of Artificial Sequence HLA-DR4 binding peptide with mutation at residues 2, 6 and 7.
FYRMVLTNPAA

SEQ ID NO: 11 - protein, 11 aa, Artificial Sequence
  Description of Artificial Sequence HLA-DR4 binding peptide with mutation at residues 2 and 6.
FWRMVMSNPAA

SEQ ID NO: 12 - protein, 11 aa, Artificial Sequence
  Description of Artificial Sequence HLA-DR4 binding peptide with mutation at residues 2 and 6.
FWRMVLSNPAA

SEQ ID NO: 13 - protein, 11 aa, Artificial Sequence
  Description of Artificial Sequence HLA-DR4 binding peptide with mutation at residues 2, 6 and 7.
FWRMVMTNPAA

SEQ ID NO: 14 - protein, 11 aa, Artificial Sequence
  Description of Artificial Sequence HLA-DR4 binding peptide with mutation at residues 2, 6 and 7.
FWRMVLTNPAA

SEQ ID NO: 15 - protein, 10 aa, Artificial Sequence
  Description of Artificial Sequence HLA-DR4 binding peptide with mutation at residue 5.
FRMVMSNPAA

SEQ ID NO: 16 - protein, 10 aa, Artificial Sequence
  Description of Artificial Sequence HLA-DR4 binding peptide with mutation at residue 5.
FRMVLSNPAA

SEQ ID NO: 17 - protein, 10 aa, Artificial Sequence
  Description of Artificial Sequence HLA-DR4 binding peptide with mutation at residues 5 and 6.
FRMVMTNPAA

SEQ ID NO: 18 - protein, 10 aa, Artificial Sequence
  Description of Artificial Sequence HLA-DR4 binding peptide with mutation at residues 5 and 6.
FRMVLTNPAA

SEQ ID NO: 19 - protein, 9 aa, Artificial Sequence
  Description of Artificial Sequence HLA-DR4 binding peptide with mutation at residue 5.
FRMVMSNPA

SEQ ID NO: 20 - protein, 9 aa, Artificial Sequence
  Description of Artificial Sequence HLA-DR4 binding peptide with mutation at residue 5.
FRMVLSNPA

SEQ ID NO: 21 - protein, 9 aa, Artificial Sequence
  Description of Artificial Sequence HLA-DR4 binding peptide with mutation at residues 5 and 6.
FRMVMTNPA

SEQ ID NO: 22 - protein, 9 aa, Artificial Sequence
  Description of Artificial Sequence HLA-DR4 binding peptide with mutation at residues 5 and 6.
FRMVLTNPA

SEQ ID NO: 23 - DNA, 39 bp, Artificial Sequence
  Description of Artificial Sequence HLA-DR4 binding peptide encoding SEQ ID NO 1.
aatttcttccgcatggtcatgtcaaacccagcggcaact

SEQ ID NO: 24 - protein, 16 aa, Homo sapiens
VNFFRMVISNPAATHQ

SEQ ID NO: 25 - protein, 13 aa, Homo sapiens
NFFRMVISNPAAT

SEQ ID NO: 26 - protein, 13 aa, Artificial Sequence
  Description of Artificial Sequence HLA-DR4 binding peptide with mutations at residue 3.
NFARMVISNPAAT

SEQ ID NO: 27 - protein, 13 aa, Artificial Sequence
  Description of Artificial Sequence HLA-DR4 binding peptide with mutations at residue 2.
NAFRMVISNPAAT

SEQ ID NO: 28 - protein, 13 aa, Artificial Sequence
  Description of Artificial Sequence HLA-DR4 binding peptide with mutations at residue 5.
NFFRZVISNPAAT

SEQ ID NO: 29 - protein, 13 aa, Artificial Sequence
  Description of Artificial Sequence HLA-DR4 binding peptide with mutations at residue 9.
NFFRMVISQFAAT